(19) United States
(12) Patent Application Publication
(10) Pub. No.: US 2023/0107981 A1
(43) Pub. Date: Apr. 6, 2023

(54) A DEVICE FOR TREATMENT OF OBESITY OR DIABETES OF A PATIENT AND A METHOD FOR SELECTING SUCH A DEVICE

(71) Applicant: Baria Tek Medical, Paris (FR)

(72) Inventors: Thierry MANOS, Marseille (FR); Christophe BASTID, Marseille (FR); Philippe POULETTE, Paris (FR); Marco GARD, Borgomasino (IT); Éric SÉJOR, Nice (FR); Youssef BIADILLAH, Maisons-Laffitte (CH)

(21) Appl. No.: 17/910,183

(22) PCT Filed: Mar. 9, 2021
(86) PCT No.: PCT/EP2021/055850

§ 371 Date: Sep. 8, 2022

(30) Foreign Application Priority Data
Mar. 9, 2020 (EP) 20315034.7

(51) Int. Cl. A61F 5/00

(52) U.S. Cl. CPC A61F 5/0036; A61F 5/0033; A61F 5/0076; A61F 2005/002

(57) ABSTRACT

A device (10) for treatment of obesity or diabetes comprises a duodenal tube (12), first anchor (14) arranged at a pre-defined distance (D) from a proximal end (15) of the duodenal tube (12). The first anchor (14) is adapted for anchoring the tube (12) distally to the pylorus (P) without mucosal involvement. A second anchor (17) in the form of a conical, inflatable balloon is used for anchoring the device (10) in the stomach (S) of the patient. The duodenal tube (12) is flexible and can be arranged in the duodenum (D) and in the jejunum (J) of the patient.

### FIELD OF THE INVENTION

The invention is directed to a device for the treatment of obesity or diabetes of a patient and to a method for selecting such a device.

### BACKGROUND TO THE INVENTION

It is known in the prior art to use implantable devices for the treatment of obesity which bypass a certain length of the duodenum. Typically such devices may be delivered in a minimally invasive manner and are anchored at one or more positions.

US 2018/0214293 discloses the anchoring of a device on the pylorus with stents or alternatively by using an inflatable balloon. A similar anchoring is disclosed in U.S. Pat. No. 9,421,116.

WO 2014/195954 discloses a device with several anchors. First and second anchors comprise stents and are pushed against the walls of parts of the duodenum. An additional, intragastric anchor for deployment within the stomach of the patient is disclosed. WO 2012/087669 also discloses intragastric anchors.

US 2005/273060 discloses the anchoring of a device on the pylorus with balloons which also reduce the volume of the stomach.

U.S. Pat. No. 5,820,584 also discloses the anchoring of a device on both sides of the pylorus. The anchoring of a device for treatment of obesity and diabetes on the pylorus by means of inflatable anchors is also disclosed in US 2011/0004320.

US 2017/312112 discloses a transpyloric device for accepting chyme from the stomach and conducting said chyme in a bypass like manner through a patients duodenum. The device is held in place by balloon segments which sit on a transpyloric conducting element.

The devices according to the prior art do all have certain dis-advantages. In particular, they may be difficult to deploy and/or anchor, they may create an undesirable mucosal engagement and they may have unreliable anchoring.

### SUMMARY OF THE INVENTION

It is an object of the present invention to overcome the drawbacks of the prior art, in particular to provide a device for treatment of obesity or diabetes which is easy to manufacture, easy to deploy, which offers a reliable anchoring and which does not have negative side effects, in particular due to mucosal engagement. A further object of the invention is to provide a method which allows to improve the treatment of the patient.

According to the present invention, these and other objects are solved with a device and a method according to the independent claims.

According to the invention, a device for treatment of obesity or diabetes is provided. Typically, with some minor modifications, the same principle of a device can be used for both, the treatment of obesity and the treatment of diabetes. The device comprises a duodenal tube. This tube is adapted to be placed in the duodenum and optionally in the jejunum or also in the ileum of a patient. A first anchor is arranged at a pre-defined distance from a proximal end of the duodenal tube. This first anchor is preferably adapted for anchoring the tube distally to the pylorus without substantial mucosal involvement, in particular without mucosal penetration. In this context, without substantial mucosal involvement means that even though the mucosa may occasionally or temporarily be contacted by the first anchor, anchoring of the device is not based on a contact, engagement or penetration of the mucosa, for example, by the anchor. More particularly, the anchor is formed and/or configured in such a way that it does not continuously and tightly contact the mucosa. By providing such an anchor, irritations of the mucosa may be avoided. Typically, the first anchor is designed such as to provide a certain weight which will create a positioning and anchoring due to peristatic effects. Since anchoring is not due to frictional engagement, the size and shape of the first anchor may be chosen such as to avoid mucosal engagement. Additionally or alternatively, another way of avoiding substantial mucosal involvement is to provide a buffer material, for example a flexible cover, around at least part of the anchor. In use, the buffer material, where used, may separate the anchor from direct contact, engagement or penetration of the mucosa. The buffer material may provide a smoother or more atraumatic tissue-facing surface than the surface of the anchor alone. The buffer material may, for example, be of different material from the first anchor. Additionally or alternatively, the buffer material may, for example, be of or comprise one or more of: polyurethane; silicone; pericardial tissue or other biological material; dacron; polytetrafluoroethylene.

In a preferred embodiment the first anchor may comprise or may be formed of an expandable structure. Typically, self-expandable structures are known in the art for providing stents. The self-expandable structure can be made of a metal, preferably of a shape memory metal such as Nitinol. Shape-memory plastics may also be used. The self-expandable structure preferably can be braided. Other embodiments such as embodiments using self-expandable structures which are cut from a tube are also conceivable. It is also possible to use balloon expandable structures or structures which are self-expandable to a certain extent, but need balloon support for a complete expansion. Other biocompatible materials such as other metals, alloys or biocompatible plastic materials are possible. In some embodiments, the expandable structure (e.g. self-expandable structure) is tubular and/or is elongate in an axial direction of the duodenal tube.

The expandable structure may be covered by one or more layer of a covering material, in particular a polymeric layer including or made of Dacron or a biologic layer, such as pericardial tissue. The layer may be a foil of a plastic material. It may also be a woven or knitted structure of polymeric filaments. The covering material may act as a buffer between the expandable structure and the mucosa to avoid substantial mucosal involvement between the anchor and the tissue, whatever the shape of the expandable structure.

The first anchor and in particular the first expandable structure may be retrievable and repositionable. A retrievable structure may be brought back into a narrower and typically into the initial configuration, allowing removal of the structure and of the entire device from the patient's body. This allows easy removal of the device, e.g. in case of unexpected side effects. The structure and hence the device also can be repositionable, i.e. allowing reduction of the size allowing to displace the device at the application site, followed by another expansion for anchoring at another site.

Any mechanism for bringing the structure and the device into a narrower configuration may be possible: Interior engagement members may allow engagement with an internal tool for crimping from the inside. Deactivation of a support structure such as an inflated balloon may lead to a collapse of an expanded structure in case the structure was expanded against a radial force, e.g. an elastic deformation. It is also conceivable to use thermal or chemical mechanisms for bringing the structure back to the narrower configuration.

It is also conceivable to additionally or alternatively anchor the duodenal tube by means of patches which are attachable to the mucosa and which are linked to the duodenal tube through connections, e.g. threads, fibres or wires. The patch preferably comprises a biocompatible adhesive. It alternatively or additionally may be adapted to promote cell biocolonization. The patches also can be biodegradeable. Such patches may help to further anchor the device, without having, however, any traumatic effect on the mucosa.

The layers and/or the patches may be formed such as to avoid endothelialisation.

According to a particularly preferred embodiment of the invention, the duodenal tube may be provided with a second anchor which is arranged at its proximal end. The second anchor is adapted to be positioned proximally of the pylorus for anchoring the device in the stomach. Optionally, the size and/or the shape and/or configuration of this second anchor may be chosen such as to avoid mucosal engagement.

The second anchor may be formed as, or comprise, a balloon (e.g. an inflatable balloon) which is adapted to be positioned within the patient's stomach such as to reduce the gastric functional volume. The balloon may be shaped and/or sized and/or configured such as to avoid a tight contact with the inner wall of the stomach when it is appropriately inflated.

As used herein, the term “balloon” is intended to cover any flexible bladder or pouch that can sealingly envelope a certain quantity of fluid, such as gas (e.g. air or nitrogen) or liquid (e.g. saline). The term “inflatable” and the like refers to the balloon being at least partly fillable with fluid to at least partly distend the balloon, whether the balloon is filled completely to capacity or only partly filled. In some embodiments, only partly filling (or partly inflating) the balloon may be a manner of configuring the balloon to avoid substantial mucosal involvement. A partly filled balloon may be more flexible and conformable against the stomach wall when subjected to stomach muscle contractions, than a balloon that is fully distended by filling to capacity (or inflating to capacity).

In a first variant, the balloon may have an annular shape surrounding the duodenal tube. Thereby, a homogenous anchoring may be achieved. Furthermore, a regular, annular shape which completely surrounds the duodenal tube may lead to regular closure pattern of the passage into the duodenum.

In a first variant, the balloon may have a crown shape when it is inflated.

In a particularly preferred variant, the balloon may have a toroidal shape.

According to another variant, the balloon may have, in the inflated condition, a specific shape and size in an area neighbouring the connection to the duodenal tube: the inflated balloon may e.g. have a conical outer shape or a concave outer shape in a cross-section through a plane running through an axis of the device. The balloon may also have a tulip shape. These shapes combine a reliable anchoring and avoid a distal migration of the device without, however, continuously requiring an engagement of the anchor with tissue and in particular mucosal engagement.

According to another variant, the second anchor may comprise one or more balloons defining plural chambers or bodies. The bodies may be independently inflatable, or they may be interconnected to be in fluid communication with one another. The bodies may optionally be defined by respective plural balloons and/or by at least a first balloon segmented or partitioned into plural bodies. The bodies may have one of more shapes selected from spherical, and/or tear-drop, and/or any other desired form. The bodies may nestle together to define collectively a voluminous bulb, e.g. with a tulip shape, but with a fluted or lobed exterior profile presenting a smaller tissue-contacting extremity than would a smooth bulbous single body. The spaces between and around adjacent bodies also help to keep open natural passages to allow chyme to enter the duodenal tube, and avoid trapping of chyme outside the duodenal tube at the antrum.

By using a conical, concave or tulip shape, and/or by using one or more balloons defining plural bodies, it is possible to place the device closer to the pylorus without the risk of contacting tissue.

Typically, the balloon and/or collectively the multiple bodies if used, may be adapted to be inflated to a volume of between 200 ml to 800 ml, more preferably to 300 ml to 450 ml.

While it is understood that such kind of second anchors are particularly preferred in combination with a device with a first anchor as described herein above, the skilled person will appreciate that such second anchor also can be used without such first anchor or with a differently shaped first anchor.

The second anchor additionally or alternatively may comprise a second expandable, in particular self-expandable structure. Such a device in particular may decrease the absorption of sugars and lipids and may be particularly suitable for treatment of diabetes.

The second anchor may have an at least partial hour-glass shape so that, preferably together with the first anchor, there may be an hour-glass like shape for anchoring on both sides of the pytorus. The hour glass shape may be symmetric or asymmetric, for example, with respect to a shape on either side of the pylorus, and/or with respect to a shape around the longitudinal axis of the tube.

Also this expandable structure may be made of a metal, preferably a shape memory metal such as Nitinol. Also the second expandable structure may be braided or alternatively laser-cut from a metallic tube. The second expandable structure also may be covered by one or more layers, in particular a polymeric layer including or made of Dacron or polyurethane or a biologic layer, such as pericardial tissue. The layer may be a foil of a plastic material. It may also be a woven or knitted structure of polymeric filaments. The layer may act as a buffer to avoid substantial mucosal involvement between the expandable structure and the stomach tissue.

The self-expandable structure of the second anchor may include at least a tubular portion. In some forms, the self-expandable structure flares outwardly in a direction away from the distal end of the duodenal tube. The flared shape may, for example, be conical or at least partly curved (e.g. curvilinear). The flared shape may, for example, resemble any of a tulip, an umbrella, a dish, or a trumpet mouth. The flared portion may, for example, comprise a plurality of arms or ribs extending from a hub. Alternatively, the flared shape may comprise a lattice structure or a braid.

In some embodiments, the second anchor may comprise both a self-expandable structure and at least one balloon. The self-expandable structure may optionally be attached to the balloon. The self-expandable structure may be arranged outside the balloon, or in a fluid chamber of the balloon, or in an open space around which the balloon is disposed, or in a wall of the balloon. The self-expandable structure may extend circumferentially around a portion of the duodenal tube adjacent to or at the proximal end of the duodenal tube.

The self-expandable structure may serve to hold open the duodenal tube and/or a passage within the balloon, for the evacuation of stomach contents, and resist any tendency for the tube or the passage to be permanently crushed or constricted under the inflation pressure exerted by the surrounding balloon. The self-expandable structure may also deform temporarily in response to stomach contraction forces, but return towards its expanded state when the stomach contraction relaxes. Additionally or alternatively, the self-expandable structure may serve to bias the balloon towards a predetermined expanded shape before and/or after inflation of the balloon.

The self-expandable structure and the balloon may be generally co-extensive at least in one axial direction with respect to an axis of the duodenal tube, optionally in both axial directions. Additionally or alternatively, one of the self-expandable structure and the balloon may extend proximally of or proximally beyond the other. For example, the balloon may extend proximally beyond an end of the self-expandable structure and/or proximally beyond a proximal end of the duodenal tube.

Optionally, only one of the two anchors comprises both a self-expandable structure and a balloon. This can reduce the amount of superimposed material necessary to fold and/or compress to a compressed state for introduction. The other anchor may comprise selectively only an expandable structure (e.g. a self-expandable structure) or a balloon.

For example, in some embodiments, only the second anchor comprises the combination of a self-expandable structure and a balloon. There is more space for such an anchor in the stomach, than in the duodenum. Also, since the need for fluid inflation adds complexity to the delivery system, for example with a fluid inflation conduit and removable connection, including these features on only the stomach side, avoids over-complicating the delivery system, particularly in the region of the duodenum where there is less space. It also simplifies the procedure for introducing and installing the duodenal tube in a patient.

In a similar way as described in context with the first anchor, also the second anchor may be retrievable and/or repositionable. The second anchor may also comprise patches, similar as described above in context with the first anchor.

According to a preferred embodiment of the invention, the device may be provided with at least one element on the duodenal tube in addition to the anchors. Such element may have several purposes. It can be used for imaging if it is of metal or other radiopaque material and provides a higher contrast in e.g. x-ray imaging or in a CT scan. If made from metal or other materials with a high specific weight, it can also be used for additionally anchoring the device due to its weight.

In one embodiment the at least one element is metallic and can be formed as a ring which is mounted on the surface of the duodenal tube. Typically, the ring can be mounted at the distal end of the duodenal tube. Additionally or alternatively it can also be mounted in an area of the duodenal tube which is distant from the distal end. Typically, it can be arranged at a distance of 8 to 12 cm from the distal end of the duodenal tube.

Additionally or alternatively to any of the above, the duodenal tube may comprise reinforcement for resisting any tendency of the duodenal tube to twist, at least in one or more local regions. Twisting of the tube can create kinks that might narrow the tube and, in severe cases, completely block the tube itself to all passage of stomach contents through the twisted region. In one variant, reinforcement of the duodenal tube may be provided between the first and second anchors. The reinforcement may, for example, comprise structure, struts or filaments in or on the tube, optionally extending from one or both anchors. Additionally or alternatively, reinforcement may be provided in a portion of the tube that is distal to the first anchor. The reinforcement may again comprise structure, struts or filaments in or on the tube, optionally extending in a spiral along an axis of the tube. In either case, some of the structure, struts or filaments may extend in a direction that is at least partly axial, to buttress the tube against twisting. The structure, struts or filaments may be of metal, for example, nitinol or stainless steel, or of plastics, for example, PET or polyurethane or polytetrafluorethylene.

According to still another embodiment of the invention, the duodenal tube may be adapted to be shortened for adaptation to at least one characteristic of the patient. For this purpose, the tube in particular can be provided with markings which indicate a certain length and/or with weakening zones which facilitate shortening. Depending on certain characteristics of the patient to be treated, shorter or longer tubes may be selected for implantation. The characteristics typically may be the thickness of abdominal fat panicle, the fat mass surrounding the abdominal cavity, the visceral fat mass or the fat inside and/or outside the abdominal cavity. Computerized Tomography may be used in a non-invasive, objective and easy to repeat evaluation. Other characteristics may be the body surface area, the body mass index or the abdominal perimeter. By providing such an adaptation, an optimal treatment may be achieved. In particular, the amount of the effect created by the bypass may be individually chosen.

According to another preferred embodiment of the invention, the device can be activatable in dependence of a contact with the content of the stomach and or intestine. For this purpose, at least one of the duodenal tube, the first and the second anchor may be activated, e.g. expanded, if brought in contact with body fluid or nutrients. In particular, the duodenal tube may be dilated in response to a contact with contents of the stomach or intestine. In particular, the device hence may only become dilated if needed, i.e. if the stomach or intestine is filled.

Typically, the first anchor has an axial length of 1 to 10 cm, optionally 1 to 3 cm. Typically, the second anchor has an axial length of 2 to 10 cm.

The duodenal tube may have a length in the range of 300 to 800 mm, preferably 400 to 700 mm. It may have a diameter in the range of 20 to 35 mm. In a particularly preferred embodiment, the tube has a length of about 600 mm and a diameter of about 28 mm.

Preferably, the duodenal tube is made of a material which avoids a contact of nutrients migrating within the tube with the duodenal wall. Typically, the duodenal tube may be made of polyurethane. Any other material having a suitable anti-migrating effect on nutrients may be used. Furthermore it is possible to select a material for the duodenal tube which is reactive to the content of the stomach or intestine: In particular the material of the duodenal tube may have an osmotic permeability for specific nutrients which may decrease the higher the content of the nutrients is. It is also possible to choose a material which selectively reduces the retention or absorption of certain constituents within the duodenum. The material may e.g. be selectively permeable for fats, proteins or sugars, depending on the desired treatment.

According to another preferred embodiment, the duodenal tube may include sensors and/or actors (e.g. actuators) for actively modifying the structure and/or the shape and/or the size of the duodenal tube in reaction to changing conditions. In particular, there may be actor on the device which modifies the shape of the tube in answer to the amount or type of bodily fluids measured by the sensors. For this purpose, the device may include electronics for treatment of signals provided by the sensors and for controlling an actuator, e.g. voltages influencing the structure of the material of the duodenal tube or driving members for changing the shape or the size such as e.g. piezo electric elements.

Another aspect of the invention provides a device (for example, a duodenal tube optionally having any of the features discussed above) for installation in the gastrointestinal tract of a patient for the treatment of obesity or diabetes, the device having one or more sensors, for example one or more biosensors, which provide information, e.g. relating to such as the content of nutrients or the status, shape or size of the device. Such information may be used within the device in a closed loop feedback and/or may be transmitted externally, e.g. via wireless communications for subsequent use by a care person. In particular, it may be possible to selectively change the size and shape of the device in answer to the amount of nutrients measured in the stomach and/or in the intestine. In particular, in case of a higher amount of nutrients, the volume occupied by the device and/or the surface of the stomach and/or the intestine covered by the device may be increased.

In some embodiments, the sensor may be a pressure sensor for measuring inflation pressure within a balloon. The balloon may, for example, be a balloon of one or both anchors of a duodenal tube as discussed above. Additionally or alternatively, the balloon may be a balloon that is installed in the stomach. The measured pressure may be transmitted externally to an external monitor or display (for example, a wrist-worn or hand-held portable electronic device).

Transmission of data between the device and an external monitor may be via any wired or wireless communication path, such as a near-field wireless communication technique that also enables power to be transmitted to the sensor, for example, by means of an inductive coupling or a radio-frequency communication coupling.

While it is understood that specific adaptive or selective materials or mechanisms or sensors for the device and in particular the duodenal tube are particularly advantageous in context with a device with anchors as described herein above, it is understood that such materials or mechanisms can be used in context with any other implantable device for treatment of obesity or diabetes, installable in the gastrointestinal tract. The device may optionally be adapted to be placed in the stomach and/or may have a duodenal tube adapted to be placed in the duodenum and optionally in the jejunum or ileum of a patient.

Another aspect of the invention is directed to a method for selecting a device for treatment of obesity or diabetes of a patient. In particular, the method is used for selecting a device as it has been described herein above.

In a first step at least one characteristic of the patient is determined. This characteristic may be the thickness of the abdominal fat panicle, the fat mass surrounding the abdominal cavity, the visceral fat mass or the fat inside and/or outside the abdominal cavity. The characteristic may also be the body surface are, the body mass index or the abdominal perimeter, measured on a standing patient at the umbilicus. The characteristic further may be the elasticity of the stomach, an index of elasticity of the stomach or evaluation tests of absorption of nutrients. By way of this, the patient specific absorption e.g. of fats may be taken into consideration. Of course, several of these characteristics can also be used in combination with each other.

On the basis of the selected characteristic or characteristics, an appropriate length of the duodenal tube is then defined.

In a final step, a device with a duodenal tube having the defined length is provided. This can be done by either shortening of the duodenal tube to the defined length or by selecting a device having a duodenal tube with the defined length. Such selection may be made either by selecting the device out of a set of devices having a various lengths or by individually manufacturing a device having the selected length.

Alternatively or additionally to the step of defining the length of a duodenal tube, the filling volume of a gastric anchoring balloon may be defined based on the determined characteristic or characteristics.

According to a preferred embodiment of the invention, the fat inside and/or outside the abdominal cavity may be determined by computerised Tomodensitometry.

This method provides for an individualised and optimised device for treatment specifically for the individual patient.

Additionally or alternatively to any of the above, one aspect of the invention provides a device for treatment of obesity or diabetes. The device comprises a duodenal tube. This tube is adapted to be placed in the duodenum and optionally in the jejunum or also in the ileum of a patient. A first anchor is arranged on the duodenal tube, optionally at a pre-defined distance from a proximal end of the duodenal tube. This first anchor is adapted for anchoring the tube distally to the pylorus (for example, without substantial mucosal involvement). A second anchor is arranged at and/or coupled to a proximal end of the tube (for example, without substantial mucosal involvement). Each of the first and second anchors may comprise a self-expandable structure, for example, made of shape-memory material, optionally a shape-memory metal, optionally nitinol. Only one of the first and second anchors, optionally the second anchor, additionally comprises a balloon, for example, an inflatable balloon.

### BRIEF DESCRIPTION OF THE DRAWINGS

The invention will now be described with reference to certain embodiments and the accompanying drawings which show:

FIG. 1: A schematic representation of a first embodiment of the invention

FIG. 2: A schematic representation of a second embodiment of the invention

FIG. 3: A representation of a third embodiment of the invention

FIG. 4: The device according to FIG. 3 deployed within a patient

FIG. 5: A schematic representation of a fourth embodiment of the invention

FIG. 6: The device of FIG. 5 deployed in a patient

FIG. 7: A flow-chart showing the steps of a method according to the invention

FIG. 8: A schematic representation of a fifth embodiment of the invention

FIG. 9: CT images for evaluation of the abdominal fat.

FIG. 10: A schematic representation of a sixth embodiment of the invention, showing principally the anchors when in situ

FIG. 11: A schematic representation of a seventh embodiment of the invention, showing principally the anchors when in situ

FIG. 12: A schematic representation of an eighth embodiment of the invention in situ

FIG. 13: A schematic representation of a ninth embodiment of the invention in situ

FIG. 14: A schematic representation of a tenth embodiment of the invention in situ

FIG. 15: A schematic perspective view of the device from in FIG. 14, shown in isolation

FIGS. 16A-G: Schematic representations of a technique for deploying the device in a patient's gastrointestinal tract

FIG. 17: A schematic representation of an eleventh embodiment of the invention, and

FIG. 18: A schematic representation of an electronic system for monitoring the status of the device.

### DETAILED DESCRIPTION OF PREFERRED EMBODIMENTS

In the following description, the same reference numerals, where used, denote similar or corresponding features, whether or not described explicitly. The disclosure of one embodiment is thus to be read in combination for another embodiment. Different reference numerals may also be used to denote similar or corresponding features where this helps the description.

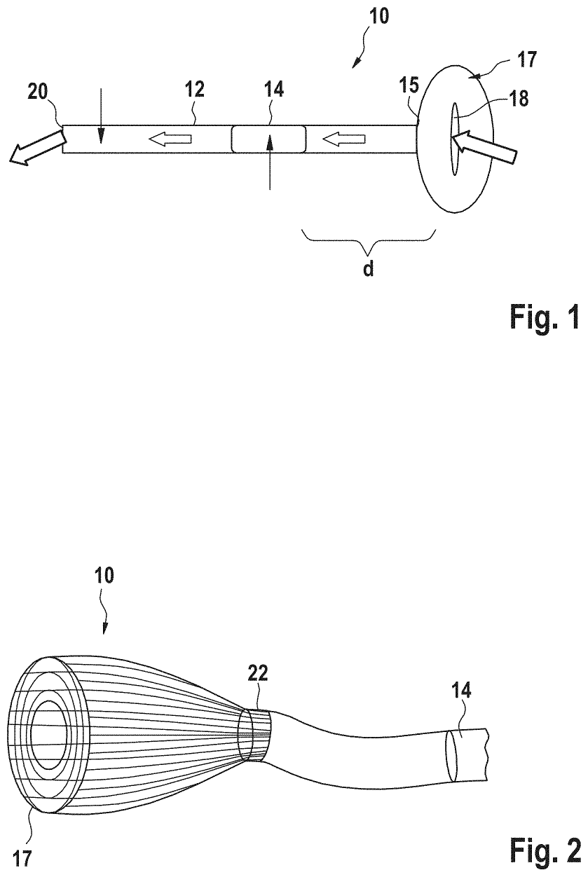

FIG. 1 discloses a first embodiment of a device 10 which is used for treating a patient suffering from obesity. The device 10 is primarily formed by a duodenal tube 12 which has a second anchor 17 attached to its proximal end 15. Arranged at a distance d from the proximal end 15 there is a first anchor 14. The first anchor 14 is formed as a mass which has a tendency to move the duodenal tube 12 distally within the duodenum. This movement can be caused by peristaltic and/or gravitational effects. Second anchor 17 avoids a too far distal migration of the device 10.

The second anchor 17 is formed in the shape of a torus and surrounds the perimeter of the duodenal tube 12 neighbouring its proximal end 15. An entry opening 18 is arranged in the toroidal second anchor 18. Nutrients may enter the interior of the duodenal tube 12 from the stomach through the opening 18, as indicated with an arrow in FIG. 1. The nutrients then quit the duodenal tube 12 at its distal end 20, again indicated by an arrow.

Thereby, a certain distance of the duodenum is bypassed and nutrients are prevented from contacting the wall of the duodenum.

In the present embodiment, the duodenal tube has a length of 600 mm and a diameter of 28 mm and is integrally formed with the balloon-like second anchor 17. It is formed of polyurethane. The mass 14 preferably is formed of a self-expandable, braided structure of wires of a shape memory material. The second anchor 17 comprises an entry opening (not shown) which is connectable with a device for inflation which allows to inflate the balloon to an appropriate volume and size, typically to a volume of 350 ml.

FIG. 2 shows an alternative embodiment of the second anchor 17. In difference to the embodiment of FIG. 2, the second anchor 17 is not formed as a torous, but rather has a tulip shape, i.e. a shape which, in a cross-section through a plane running through an axis of the device is slightly convex.

In the embodiment shown in FIG. 2, the first anchor 14 is designed as a self-expandable structure made of braided nitinol wires. An additional support structure 22 is disclosed which helps to stabilize the device and/or anchor the device.

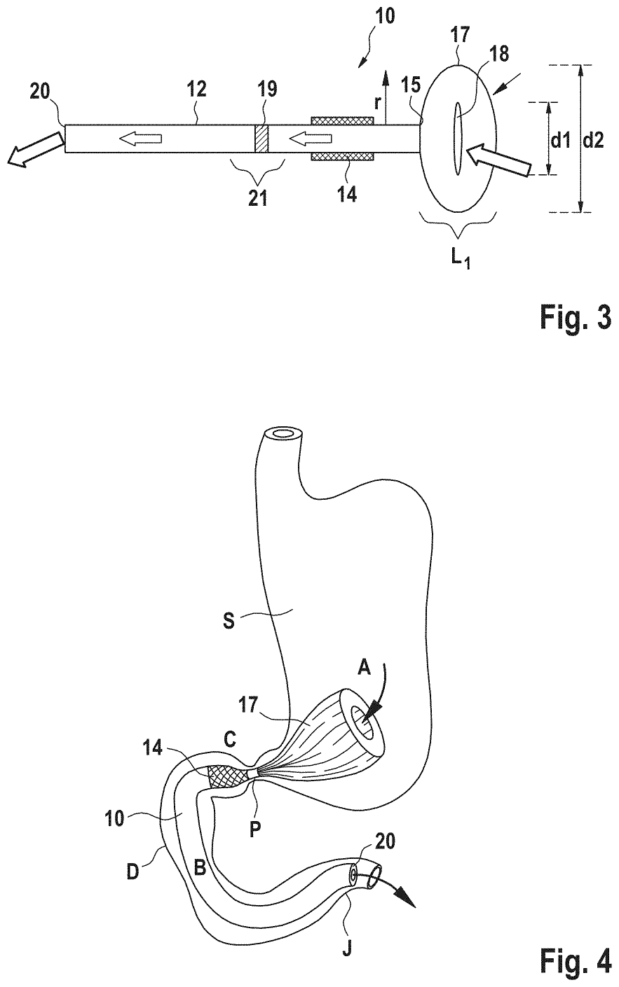

FIG. 3 shows a more specific embodiment of a device 10 for treating a patient with obesity. The device is partly similar to the device of FIG. 1, with the following differences: the first anchor 14 is formed as a self-expandable structure made of braided nitinol. The embodiment of FIG. 3 is formed of the duodenal tube 12 made of polyurethane and of the inflatable second anchor 17. The second anchor 17 is attached to the duodenal tube 2 by gluing and is made of silicon. The second anchor 17 may have a toroidal, conical or tulip shape. The second anchor 17 has an axial length 11 of about 50 mm, an internal diameter d1 of the opening 18 of about 28 mm and an external diameter d2 of about 50 mm. The lips of the second anchor 17 typically may have a radial length r of 10 to 25 mm. By appropriately selecting the size, thickness and/or material of the duodenal tube 12, it can be made sure that the tube is sufficiently flexible such as to adopt its shape to the shape of the duodenum D and optionally the jejunum J.

The device 10 deployed within the patient is shown in FIG. 4.

The device is anchored proximately of the pylorus P by means of the second anchor 17 whereas it is anchored distally of the pylorus P by means of the first anchor 14. The second anchor 17 has a conical or tulip-like shape which avoids contact with the mucosal wall of the stomach S. The second anchor 17 has a double function. On the one hand it reduces the volume of the stomach for reducing the feeling of hunger of the patient. Additionally, it avoids migration of the device into the distal direction. Due to the conical or tulip shape, a permanent contact with the mucosal wall is, however, avoided. The second anchor 17 may freely float within the stomach. The first anchor 14 is also sized and shaped such that mucosal contact can be avoided. It is made sufficiently short, typically with a length of 10 mm to 25 mm and covered by a layer of, for example, a buffer material, made of a biocompatible material such as Dacron and having a thickness of 0.7 mm to 2 mm, which is not shown in detail in FIGS. 3 and 4.

In addition, in the embodiment shown in FIG. 3, the duodenal tube 12 is provided with a ring 19 of a radio opaque material, in particular a metal. The ring 19 is arranged in an area 21 of the duodenal tube 12 which is distant from the proximal end 20 of the duodenal tube 12. This helps positioning the device under x-ray control.

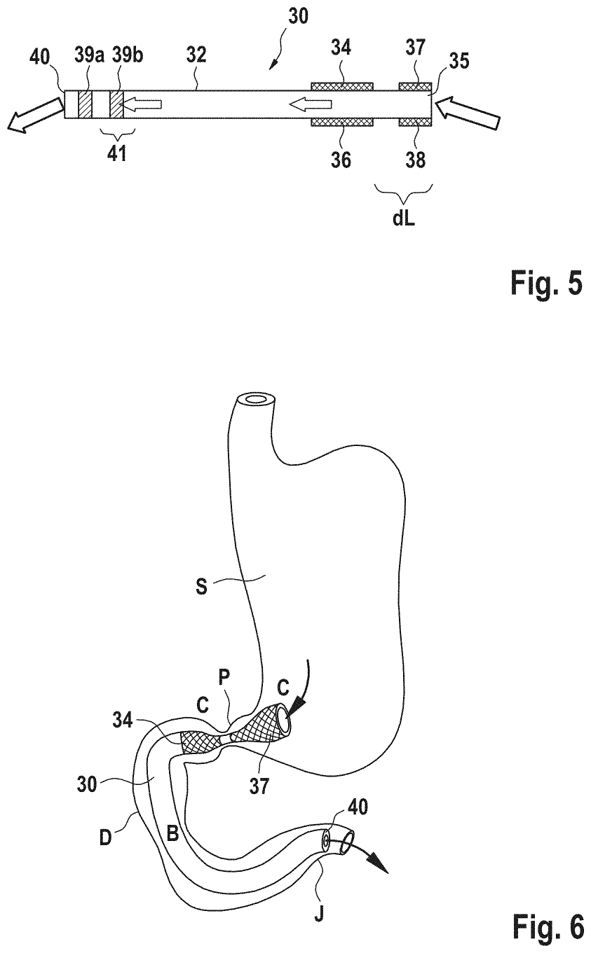

FIG. 5 shows an embodiment of the invention which is suitable for treatment of diabetic patients. The device 30 according to FIG. 5 also comprises a duodenal tube 32. The duodenal tube 32 is provided with a first anchor 34 and a second anchor 37. The second anchor 37 is arranged neighbouring the proximal end 35 of the duodenal tube 32 whereas the first anchor 34 is arranged at a distance d of the proximal end 35. The first anchor 34 and the second anchor 37 both are formed of expandable structures 36, 38 of braided nitinol wire, respectively.

The duodenal tube 32 is provided with two rings on its outer surface: a first ring 39a is arranged at the distal end 40 whereas a second ring 39b is arranged in an area 41 which is distant from the distal end 40. Similar as in the embodiment of FIG. 3, the duodenal tube 32 of FIG. 5 is made of polyurethane. The anchors 34, 37 and the rings 39a, 39b are mounted on the duodenal tube by gluing. It is, however, also conceivable to integrate in particular the rings 39a, 39b between multiple layers of the duodenal tube.

In the embodiment shown in FIG. 5, the second ring 39 is typically arranged at the distance of 10cm from the distal end 40.

FIG. 6 shows the device 30 of FIG. 5 deployed within a patient. The first and second anchors 34, 37 are arranged on both sides of the pylorus and prevent distal or proximal migration of the device while still avoiding a direct contact with the tissue of the involved stomach S or the duodenum D. The anchor 37 is substantially smaller that the anchor 17 of the embodiment of FIGS. 3 and 4, making the device 30 of FIGS. 5 and 6 less suitable for treatment of obesity, but suitable for diabetes treatment.

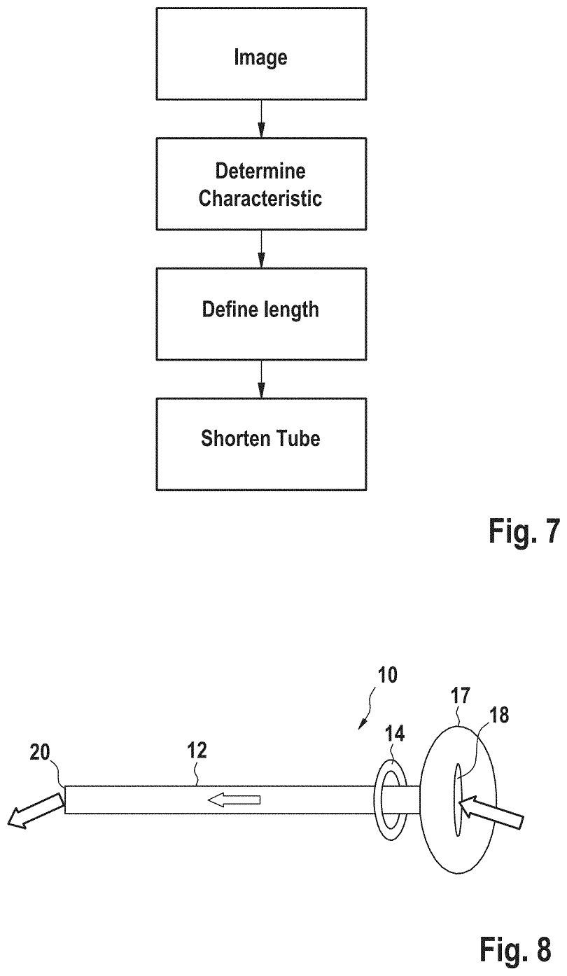

FIG. 7 schematically shows various steps for selecting a device which is appropriate for an individual patient. In a first step “image” the patient is examined with examination methods known to the skilled person, in particular by diagnostic imaging. Based on this examination or imaging step, certain characteristics of the patient are then determined in step “Determine characteristic”. Based on a CT scan, the thickness of the abdominal fat panicle, the fat mass surrounding the abdominal cavity and the visceral fat mass is determined. In addition, the body surface area, the body mass index and the abdominal perimeter are clinically defined. Additionally, it is also possible to define by way of CT scan the fat inside and/or outside the abdominal cavity. Based on these six or seven objective criteria, the length of the duodenal tube is defined in step “Define length”. The length is typically between 450 and 600 mm. As a general rule and by way of example, for more severe conditions of obesity, longer tubes will be chosen.

The first three criteria easily can be determined manually on the basis of an image shown on a display screen of a CT scanner based on a cross-section along the third lumbar vertebra. Of course these criteria also can be assessed fully or partly automatically by using artificial intelligent software.

In a final step “Shorten Tube”, the tube is shortened to the defined length. Shortening made be made by cutting. For that purpose, markings may be provided on the outer surface of the duodenal tube (not specifically shown in FIG. 3). A ruler can be associated to the device.

Alternatively to the step of shortening the tube, a tube with an appropriate length may be chosen from a set of standard dimensions or may be individually manufactured.

Additionally or alternatively to the step of defining the length, also the inflation volume of the balloon like second anchor 17 may be determined based on the characteristic(s). By inflating the balloon to different volumes, an additional adaptation to an individual patient is possible. The amount to which the stomach should be filled and the size of the second anchor thereby may be defined.

FIG. 8 shows another embodiment of a device 10 similar to the one shown in FIG. 1. In the embodiment of FIG. 8, the first and the second anchor are formed by inflatable balloons 14, 17.

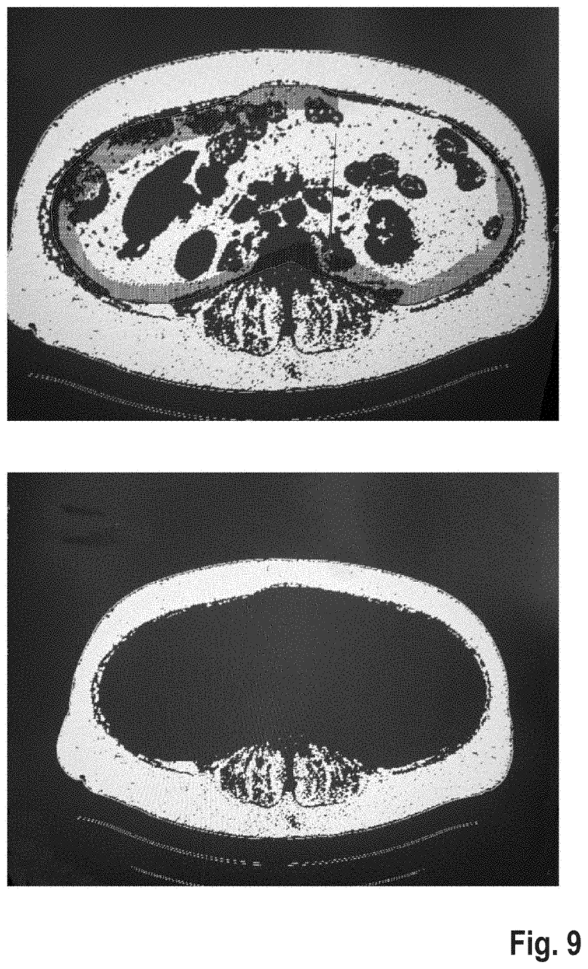

FIG. 9 shows two CT images. The abdominal fat is evaluated using the CT images and variations of the grey scale.

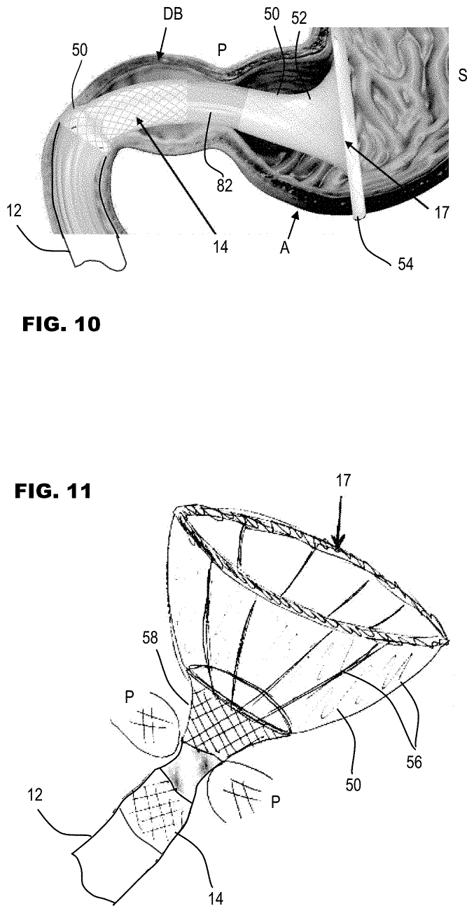

FIGS. 10 to 17 show further embodiments of a device 10 similar to the preceding embodiments. The embodiments of FIGS. 10 to 17 are similar to one another in that the device 10 comprises a first anchor 14 mounted on the duodenal tube 12. The first anchor comprises a self-expandable structure, optionally in the form of a stent-like structure made of braided wires. The first anchor 14 may be made of shape memory material, for example, nitinol. The first anchor 14 can be covered with a buffer material 50, for example, of polyurethane. The configuration of the first anchor 14 and/or the provision of the buffer material can avoid substantial mucosal involvement between the first anchor 14 and the duodenal mucosa.

For example, as can be seen in FIG. 10, the first anchor 14 is dimensioned to bridge the duodenal bulb DB without substantial mucosal involvement. The first anchor 14 may have a length of between about 50 mm and 100 mm, for example, about 80 mm. The first anchor 14 may have a diameter (for example, between about 20 mm and 40 mm, for example about 25 mm or 30 mm) such that the first anchor 14 resists any tendency for the duodenal tube to shift proximally through the pylorus towards the stomach S. The first anchor 14 may include a distal shoulder (for example, of about 40 mm in diameter) to provide an additional stop.

The embodiments of FIGS. 10 to 17 differ from one another in the implementation of the second anchor 17. In the embodiment of FIG. 10, the second anchor 17 comprises a self-expanding structure 52, similar to FIG. 6. However, in FIG. 10, the self-expanding structure has a shape like a trumpet mouth, with a proximal end forming a flange 54. The self-expanding structure 52 may have a braided or lattice structure made of a shape-memory material for example, nitinol. The self-expanding structure is covered with a buffer material 50, for example, silicone or polyurethane.

As can be seen in FIG. 10, the second anchor 17 closes off the antrum A of the stomach. The proximal flange 54 engages the stomach wall, and defines a closed-off space behind the flange, thereby reducing the volume of the stomach available for receiving food.

In the embodiment of FIG. 11, the second anchor 17 comprises a flared shape defined by a plurality of diverging ribs or fingers 56. The fingers 56 diverge in a direction away from the distal end of the tube 12, and extend from a collar 58 at the proximal end of the tube 12. The second anchor 17 may be covered by a buffer material 50, as described above with reference to FIG. 10.

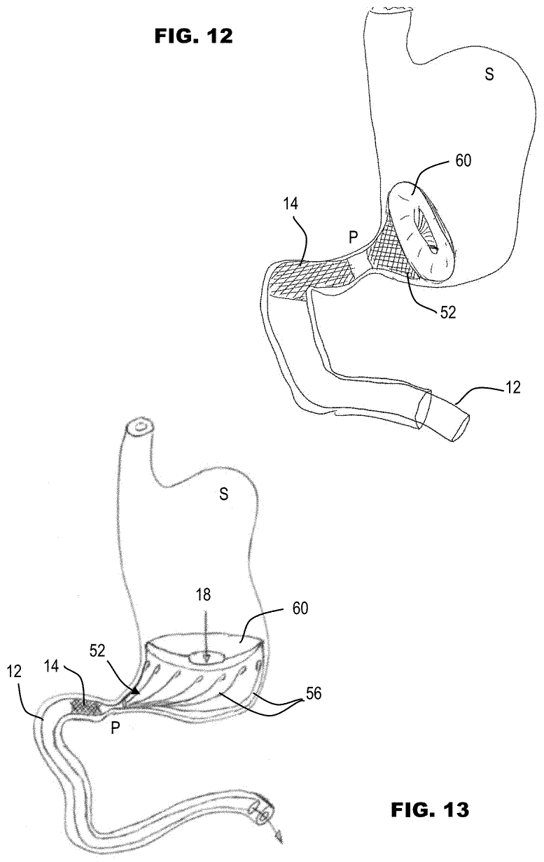

In the embodiment of FIGS. 12 to 17, the second anchor 17 comprises a self-expandable structure 52 and a balloon 60, for example, an inflatable balloon. Various implementations are envisaged. Where a self-expandable structure 52 and balloon 60 are used together, it is optionally preferred that this be for only one of the anchors, in the illustrated embodiments, the second anchor 17. The combination of both structures increases the amount of material used at the anchor, and may have some effect on the size to which the anchor can be collapsed or compressed for delivery. Employing the combination of structures at one anchor can help limit the impact on size compared to using such a combination for both anchors. Employing the combination of structures specifically for the second anchor 17 may be appropriate for the anatomy, because there is more space available in the stomach than in the duodenum. There may also be a greater need for anchoring from the stomach side (to resist displacement towards the intestine) than from the duodenum side (to resist displacement towards the stomach), because the natural flow of chyme, and the majority of muscular movement, is from the stomach side.

In FIG. 12, the self-expanding structure 52 has a flared shape, diverging outwardly in a direction away from the distal end of the tube 12. The self-expanding structure may comprise a braid or lattice of shape memory material, for example, of nitinol. The self-expanding structure 52 biases the anchor 17 towards an expanded state. Inflation of the balloon 60 provides additional bulk to occupy volume in the stomach and hence reduce the functional gastric volume, and to provide an atraumatic lip around the mouth to the tube 12.

In FIG. 13, the self-expanding structure 52 comprises a plurality of ribs or fingers 56 that bias the anchor towards an expanded state. Subsequent inflation of the balloon completes or fills-in the tulip shape form around a central evacuation channel, to reduce the functional gastric volume, while keeping open a central evacuation channel for entry of chime into the duodenal tube 12.

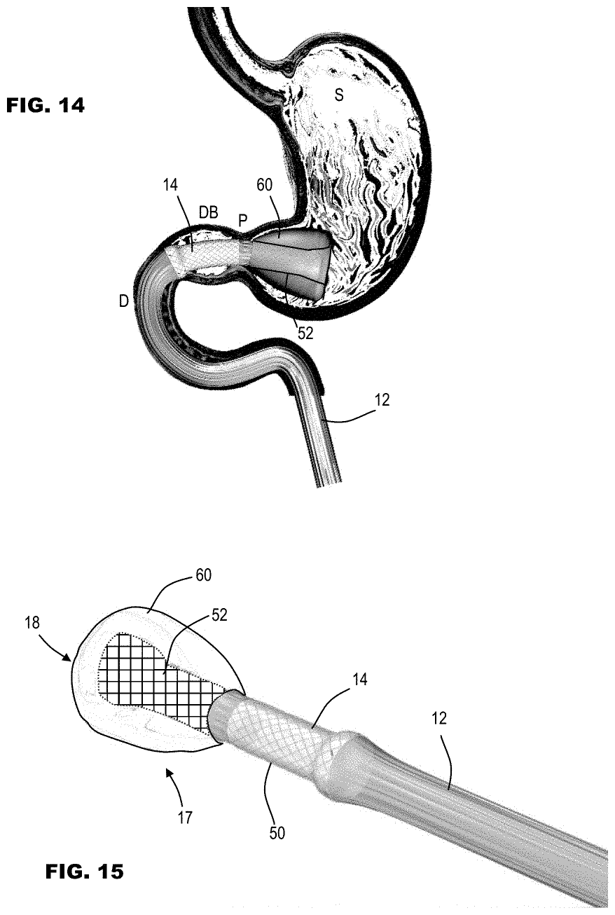

In FIGS. 14 and 15, the self-expanding structure 52 provided radially inwardly of the balloon 60, to buttress the tube 12 against the inflation pressure of the balloon 60. This can reinforce the tube 12, so that inflation pressure of the surrounding balloon 60 does not crush or collapse the tube 12, avoiding risk of blockage. However, the combination of the self-expanding structure 52 and the balloon 60 permits gastric contractions of the stomach to be transmitted to the tube 12, to advance chime into and along the tube 12 for evacuation towards the duodenum. The balloon 62 may be partly inflated, e.g. filled to less than full capacity, to facilitate transmission of the gastric contractions to the self-expandable structure 52, and to provide flexibility and conformability to the balloon 60.

The self-expanding structure 52 may be generally coextensive with the balloon 60, at least towards the proximal end of the device. The self-expanding structure 52 may comprise a tubular stent-like structure, for example a braid or lattice structure, made of shape-memory metal, for example, nitinol. In some forms, the axial length of the self-expanding structure may be between about 50 mm and 100 mm, for example about 80 mm. The diameter of the self-expanding structure 52 may be about 30 mm along a majority of its length, optionally with a flared mouth at its proximal end.

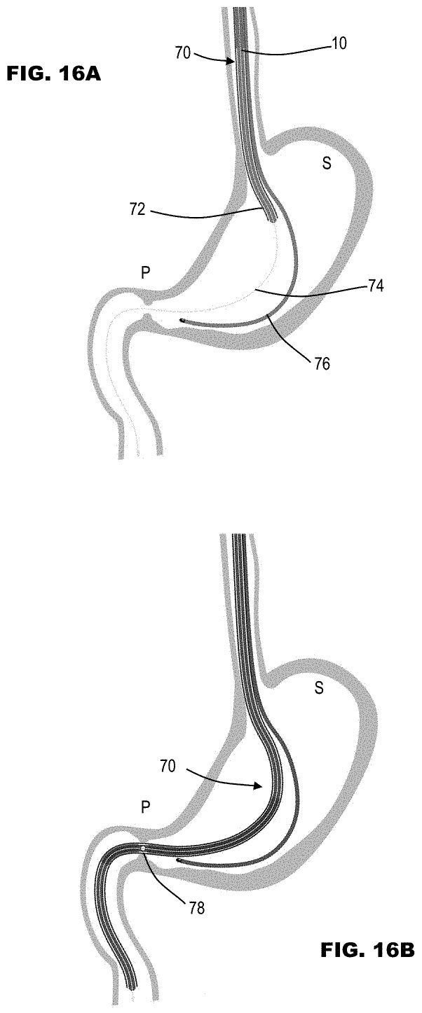

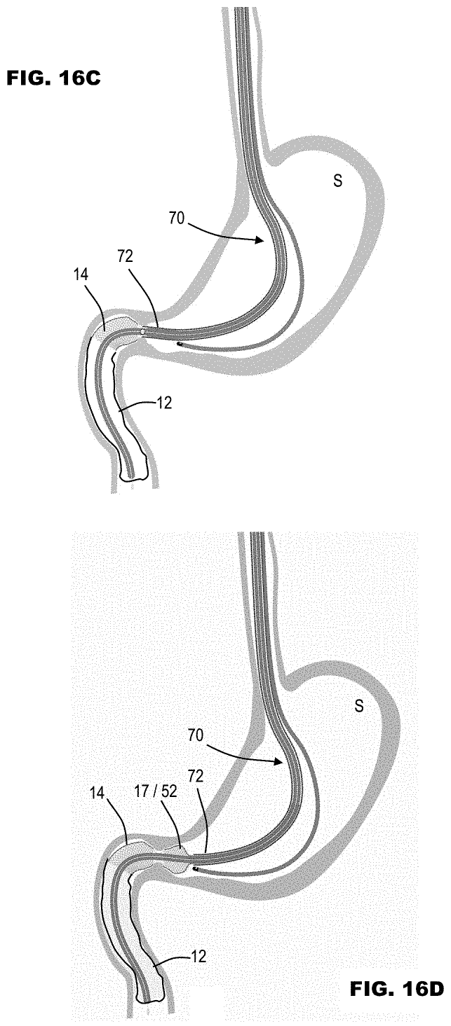

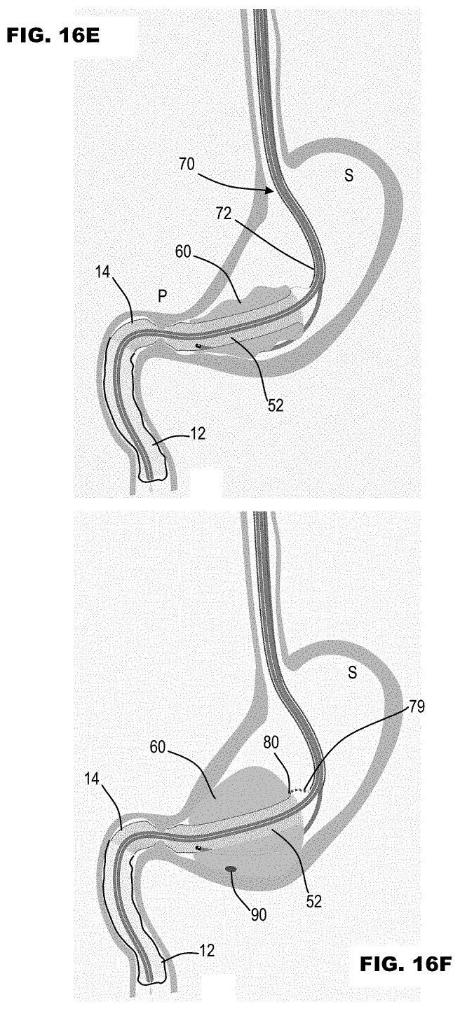

FIGS. 16a to 16g illustrate a technique for introduction and deployment of the device of FIGS. 14 and 15. Referring to FIG. 16a, the device 10 is compressed to a small size and loaded into a delivery system 70 having a sheath 72 for retaining the device 10 radially compressed. The delivery system 70 is introduced through the patient's mouth and into the stomach over a guidewire 74 placed along the gastrointestinal tract. An imaging sensor 76 may optionally provide guidance to the medical practitioner.

Referring to FIG. 16b, the delivery system 70 is advanced through the pylorus and into the duodenum, until a radio-opaque marker 78 (either on the tube 12 or on the delivery system 70) becomes aligned with the position of the pylorus.

Referring to FIGS. 16c and d, retraction of the sheath 72 deploys the duodenal tube 12 progressively from the distal end, allowing the self-expanding structures of the first anchor 14 and the second anchor 17 to expand on either side of the pylorus. Referring to FIG. 16e, complete retraction of the sheath 72 also reveals the balloon 60 which, initially, is uninflated. Referring to FIG. 16f, the balloon 60 is inflated to its operative size by means of an inflation line 79 of the delivery system 70 coupled to an inflation port 80 of the balloon 60. Referring to FIG. 16g, after inflation, the inflation line 78 is disconnected from the port 80, and the delivery system 70 then removed, leaving the device 10 in situ.

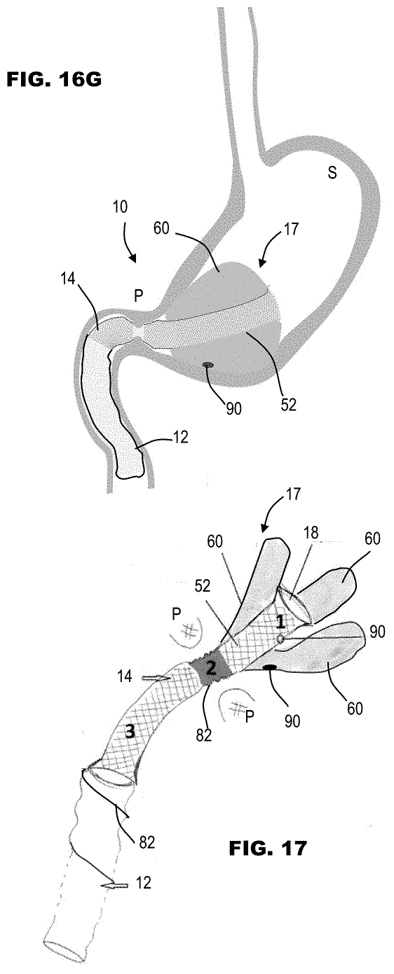

FIG. 17 illustrates a further embodiment in which the second anchor 17 comprises one or more balloons defining plural inflatable bodies 60. In the illustrated form, the bodies 60 are formed by separate balloons, although in other embodiments, the bodies 60 may be implemented as respective chambers of the same balloon structure. The bodies 60 may be independently inflatable, or the bodies 60 may be in fluid communication with one another. The bodies 60 may have one of more shapes selected from elongate and/or spherical, and/or tear-drop, and/or any other desired form. The bodies 60 may nestle together to define collectively a voluminous bulb, e.g. with a tulip shape, but with a fluted or lobed exterior profile presenting a smaller tissue-contacting extremity than would a smooth bulbous single body. The spaces between and around adjacent bodies also help to keep open natural passages to allow chyme to enter the duodenal tube, and avoid trapping of chyme outside the duodenal tube and bodies.

Optionally, the second anchor 17 further comprises a self-expandable structure 52. The self-expandable structure 52 may at least partially overlap the bodies 60, or the self-expandable structure may be shorter, such that any overlap is minor.

In all of the embodiments described herein, and whether or not illustrated in the drawings, the duodenal tube 12 may optionally comprise reinforcement 82 (e.g. FIGS. 10 and 17) for resisting any tendency of the duodenal tube to twist, at least in one or more local regions. Twisting of the tube can create kinks that might narrow the tube and, in extreme cases, completely block the tube itself to all passage of stomach contents through the twisted region. In one variant, reinforcement 82 of the duodenal tube 12 may be provided between the first and second anchors 14 and 17. The reinforcement 82 may, for example, comprise structure, struts or filaments in or on the tube, optionally extending from one or both anchors 14 and 17. Additionally or alternatively, reinforcement may be provided in a portion of the tube 12 that is distal to the first anchor 14, as illustrated in FIG. 17. The reinforcement 82 may again comprise structure, struts or filaments in or on the tube 12, optionally extending in a spiral along an axis of the tube 12. In either case, some of the structure, struts or filaments may extend in a direction that is at least partly axial, to buttress the tube against twisting. The structure, struts or filaments may be of metal, for example, nitinol or stainless steel, or of plastics, for example, PET or polyurethane or polytetrafluorethylene.

A further aspect of the embodiments described herein is the provision of at least one sensor 90 for sensing a characteristic useful for monitoring the status, shape or size of the device 10, or information about nutrients passing (e.g. passing through) the device 10. In one form, the sensor 90 may optionally monitor the status, shape or size of one or both of the anchors 14 and 17. Referring to FIGS. 16 and 17, one such sensor 90 may be a pressure sensor for measuring inflation pressure of the balloon or body 60 and/or the pressure changes transmitted through the balloon by stomach contractions. Additionally or alternatively, the sensor 90 (FIG. 17) may be sensor for sensing a parameter of the chyme passing through the tube 12, such as the flow rate.

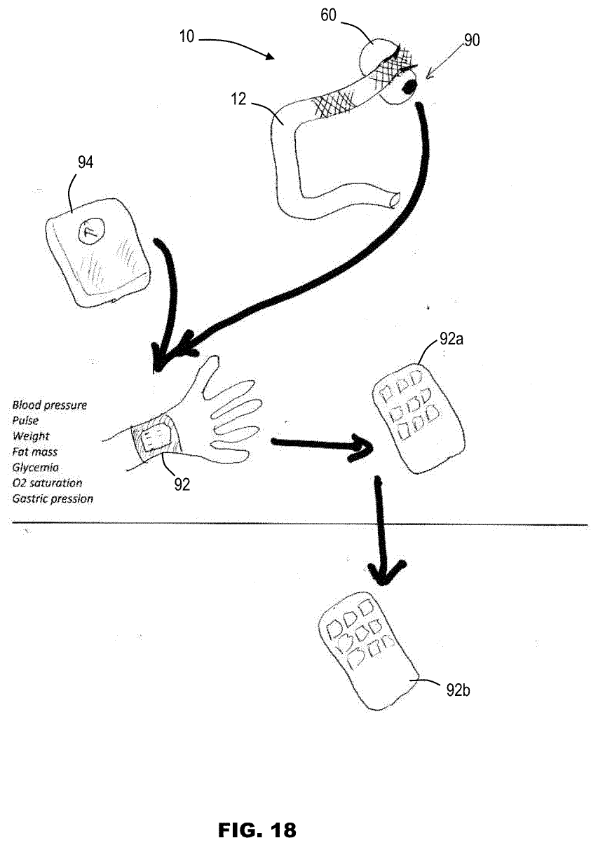

Referring to FIG. 18, a communications interface (not shown) permits the sensed information to be communicated to an external receiver and/or monitor 92, for example, a portable device such as a wrist-worn or hand-held electronic device. By way of example, the external device may be a smart-watch or a dedicated wrist-worn electronic bracelet 92. Transmission of data between the device 10 and an external monitor 92 may be via any wired or wireless communication path, such as a near-field communication technique that also enables power to be transmitted to the sensor 90, for example, by means of an inductive coupling or a radio-frequency communication coupling when the external device 92 is brought into proximity with the device 10 or its sensor 90. The communication interface may optionally be incorporated into the sensor 90 as an integrated module. The wrist-worn device 92 may further have skin-contacting sensors for measuring one or more of: blood pressure and/or blood pulse and/or blood glucose level and/or blood oxygen saturation.

Optionally, the monitoring device 92, or a partner device, such as the patient's smart-phone 92a, may include a software application for storing the information received from the sensor 90 over time, to enable the information to be transmitted to a medical practitioner's system via wired or wireless communication (for example, to the medical practitioner's smart-phone 92b) for monitoring the performance of the device 10 after installation in the patient. In the case of a device 10 intended for weight-loss, the smart-device may also receive information from weighing-scales 94 by which the patient may monitor his or her weight, and optionally body mass index, on a regular basis. In the case of a device 10 intended for treating diabetes, the smart-device 92/92a/92b may also receive information from a glucose monitor, such a skin-worn device (e.g. 92) or an electronic patch.

1.-37. (canceled)

38. A device for treatment of obesity or diabetes of a patient, comprising a duodenal tube, adapted to be placed in at least one of the duodenum, jejunum and ileum of a patient,
wherein a first anchor is arranged at a predefined distance from a proximal end of the duodenal tube, adapted for anchoring the tube distally to the pylorus.

39. The device according to claim 38, wherein the first anchor comprises an expandable structure.

40. The device according to claim 39, wherein the expandable structure is a self-expandable structure.

41. The device according to claim 39, wherein the expandable structure is made of a metal.

42. The device according to claim 41, wherein the expandable structure is made of a shape memory metal.

43. The device according to claim 41, wherein the self-expandable structure is braided.

44. The device according to claim 39, wherein the expandable structure is covered by at least one layer of material.

45. The device according to claim 44, wherein the expandable structure is covered by a polymeric layer that is at least partially made of at least one of Dacron (polyethylene terephthalate), polyurethane and a biologic layer,

46. The device according to claim 38, where the first anchor is at least one of retrievable and repositionable.

47. The device according to claim 39, where the first expandable structure is at least one of retrievable and repositionable.

48. The device according to claim 38, wherein the first anchor comprises patches attachable to the mucosa and linked to the duodenal tube via connectors.

49. The device according to claim 48, wherein the patch comprises a biocompatible adhesive.

50. The device according to claim 38, wherein the duodenal tube is provided with a second anchor at its proximal end, adapted to be positioned proximally of the pylorus for anchoring the device in the stomach without substantial mucosal involvement.

51. The device according to claim 50, wherein the second anchor is formed as an inflatable balloon, adapted to be positioned within the patient's stomach such as to reduce the gastric functional volume and shaped such as to avoid a tight contact with the inner wall of the stomach.

52. The device according to claim 38, wherein the duodenal tube is provided with a second anchor coupled to its proximal end, adapted to be positioned proximally of the pylorus for anchoring the device in the stomach.

53. The device according to claim 52, wherein the second anchor comprises at least one balloon.

54. The device according to claim 51, wherein the balloon has an annular shape surrounding the duodenal tube.

55. The device according to claim 54, wherein the balloon has a crown shape.

56. The device according to claim 51, wherein the balloon has a toroidal shape.

57. The device according to claim 51, wherein the balloon has, in an area neighbouring the connection to the duodenal tube, a conical outer shape or a concave outer shape in a cross-section through a plane running through an axis of the device.

58. The device according to claim 51, wherein the balloon has a tulip shape in the area neighbouring the connection to the duodenal tube.

59. The device according to claim 51, wherein the balloon is adapted to be inflated to a volume of 200 ml to 800 ml.

60. The device according to claim 50, wherein the second anchor comprises a second expandable structure.

61. The device according to claim 50, wherein the second anchor has an at least partly hour-glass shape.

62. The device according to claim 60, wherein the second expandable structure is made of a metal.

63. The device according to claim 60, wherein the second expandable structure is braided.

64. The device according to claim 60, wherein the second expandable structure is covered by at least one layer of material.

65. The device according to claim 38, wherein the device is provided with at least one additional element on the duodenal tube.

66. The device according to claim 65, wherein the at least one additional element on the duodenal tube is a radiopaque element.

67. The device according to claim 65, wherein the at least one element is formed as a ring mounted on at least one of a distal end of the duodenal tube and an area distant from the distal end of the duodenal tube.

68. The device according to claim 38, wherein the duodenal tube is adapted to be shortened for adaptation to at least one characteristic of the patient.

69. The device according to claim 38, wherein at least one of the duodenal tube, the first anchor and the second anchor is activatable by contact with contents of at least one of the intestine and the stomach.

70. The device according to claim 38, wherein the first anchor has an axial length of 1 cm to 10 cm.

71. The device according to claim 50, wherein the second anchor has an axial length of 2 cm to 10 cm.

72. The device according to claim 38, wherein the duodenal tube is made of a material which reduces a contact of nutrients migrating within the tube with the duodenal wall.

73. The device according to claim 38, wherein the duodenal tube has a length in the range of 300 mm to 800 mm.

74. The device according to claim 38, wherein the duodenal tube has a diameter of 25 to 35 mm.

75. The device according to claim 51, wherein amongst the first anchor and the second anchor, only one of said anchors comprises a combination of a self-expandable structure coupled to the balloon.

76. A device for treating diabetes or obesity, the device comprising a portion installable within at least one of the gastro-intestinal tract of a patient, the duodenum and the stomach, the device comprising at least one sensor which provide information relating to one or more of: (i) the status of at least a portion of the device; (ii) the shape of at least a portion of the device; (iii) the size of at least a portion of the device; (iv) nutrients passing within the device.

77. The device according to claim 76, wherein the sensor is configured for transmitting the information externally of the device via wireless communication.

78. The device according to claim 76, wherein the device comprises a balloon, and the sensor is a pressure sensor for sensing balloon pressure.

79. A method for selecting a device for treatment of obesity or diabetes of a patient, comprising the steps of:
determining at least one characteristic of the patient selected from the group of thickness of the abdominal fat panicle, fat mass surrounding the abdominal cavity, visceral fat mass, the fat inside the abdominal cavity, the fat outside the abdominal cavity, body surface area, Body Mass Index, abdominal perimeter, elasticity of the stomach, index of elasticity of the stomach and index of absorption for nutrients, measured on standing patient, at the umbilicus;
defining a length of a duodenal tube on the basis of the selected characteristic; and
providing a device with a duodenal tube having the defined length by one of (i) shortening the duodenal tube to the defined length or (ii) selecting a device having a duodenal tube with the defined length.

80. The method according to claim 79, comprising the further step of defining a filling volume of a gastric anchoring balloon based on the determined characteristic.

81. The method according to claim 79, wherein at least one of the fat inside the abdominal cavity and the fat outside the abdominal cavity is determined by Computerized Tomodensitometry.
